(19) United States
(12) Patent Application Publication
(10) Pub. No.: US 2008/0312576 A1
(43) Pub. Date: Dec. 18, 2008

(54) PLUNGER-LESS SYRINGE FOR CONTROLLING BLOOD FLOW

(76) Inventors: Robert J. McKinnon, Highlands Ranch, CO (US); James Dale Bickley, Tucson, AZ (US)

Correspondence Address: SHERIDAN ROSS PC, 1560 BROADWAY, SUITE 1200, DENVER, CO, 80202, US

(21) Appl. No.: 12/140,181

(22) Filed: Jun. 16, 2008

Related U.S. Application Data
(60) Provisional application No. 60/944,315, filed on Jun. 15, 2007.

(51) Int. Cl. A61M 5/178

(52) U.S. Cl. 604/6.15

(57) ABSTRACT

A plunger-less syringe, method of manufacture and system of use is disclosed wherein the syringe includes a barrel that has an interior receptacle for receiving a quantity of blood. The syringe has at least one filter that controls fluid flow within the syringe. The syringe may be fabricated from a material that prevents diffusion of the gas from the blood. Different syringes may comprise different fluid flow characteristics. In addition, the components of the syringe may include indicia so a syringe having particular characteristics can be selected for patients with different blood pressure.

This application claims the benefit of pending U.S. Provisional Patent Application Ser. No. 60/944,315, filed Jun. 15, 2007, which is incorporated by reference in its entirety herein.

This application is also related to U.S. Pat. No. 7,090,646, issued Aug. 15, 2006, which is incorporated by reference in its entirety herein.

### FIELD OF THE INVENTION

The present invention relates to a plunger-less syringe for receiving blood, and in particular, to such a syringe that includes a housing having an interior blood collection receptacle for drawing blood from a patient and removing the blood from the receptacle for testing. A plunger-type mechanism, bulb or other device for modifying the pressure inside the plunger-less syringe may be operably attached to the syringe when assistance is needed to draw and/or expel a patient's blood.

### BACKGROUND OF THE INVENTION

There are numerous syringes available for drawing a patient's blood. Many syringes employ a movable plunger that creates a pressure variation that assists either drawing blood into or forcing the blood from such a syringe. However, plunger-less syringes are also used to collect blood and rely predominantly on the pressure of the blood within a patient's vein or artery to fill a receptacle, such as a capillary tube as described in U.S. Pat. No. 4,393,882 to White and U.S. Pat. No. 7,090,646 to McKinnon et al., both being incorporated in their entirety by reference herein. Plunger-less syringes often include at least two openings to the blood collecting receptacle; one where the blood enters the receptacle, and one that is opposite from the location from which the blood enters the receptacle enabling air within the receptacle to exit as the blood enters. This configuration has the advantage of substantially preventing air pressure build up in the receptacle that could inhibit the flow of blood into the receptacle or that could compromise the blood sample. That is, air that contacts the drawn blood can compromise blood analysis assays, such as the determination of oxygen levels (O₂) in the collected blood, so it is desirous to expel air from the receptacle when a sample is collected.

In order to reduce or eliminate air exposure to the collected blood, various techniques are known for closing air exit openings in the blood collection receptacles of plunger-less syringes. In one technique, a filter is provided that allows air to exit the receptacle through the filter until the drawn blood saturates the filter. The filter includes a chemical that facilitates expansion of the filter material when exposed to a liquid, such as blood, thereby substantially closing the pores within the filter and preventing both blood and air from entering and exiting the receptacle. Typically, there is a time period associated with such a filter where air may pass through the filter, but the air transfer becomes more and more difficult as the pores of the filter close. Thus, there is a need to provide a plunger-less syringe in which blood may be easily or readily removed from the blood collection receptacle without concern for the passage of time.

Although plunger-less syringes may be smaller and less expensive to produce than syringes with plungers, in some circumstances plunger-less syringes lack acceptable means for controlling the movement of blood into or out of their blood collection receptacle. For example, in cases where blood is being drawn from a patient with insufficient blood pressure, additional blood drawing techniques may be required. Accordingly, it is also desirable to provide a plunger-less syringe, wherein a conventional plunger-type syringe or related pressure altering device can be attached thereto for assisting in the withdrawing of a patient's blood as may be needed. Similarly, in some situations it may also be desirable to utilize an external plunger-type syringe or similar pressure altering device to assist in expelling blood from the collection receptacle and/or in controlling the flow rate of blood exiting the collection receptacle. Still further, in situations where a filter is used and that filter subsequently becomes sealed or effectively sealed with respect to the flow of air, it is also desirable to be able to overcome the sealing or effective sealing to permit air to flow back through the collection receptacle in a controlled manner to provide control over the removal of blood from the receptacle for testing purposes.

It is another drawback of plunger-less syringes of the prior art that they are similar in structure and application and therefore do not accommodate patients with different blood pressures. More specifically, plunger-less syringes typically rely on the arterial blood pressure of a patient to provide the impetus of transferring blood from the patient into the collection receptacle within the plunger-less syringe. Depending on the arterial pressure of the patient, the velocity of blood entering into the syringe will vary. The higher the velocity, the more turbulent the blood flow will be, which may cause the capture of air bubbles from the air inside the receptacle as blood is collected. This air entrainment can adversely affect the accuracy of the test results. Conversely, if blood enters the receptacle too slowly, it will be difficult to fill the receptacle with the required amount of blood needed for testing. To address this latter issue, a medical technician may employ a plungered syringe interconnected to the plunger-less syringe to assist drawing the blood from the patient. Thus it is a long felt need to provide a plunger-less syringe that addresses issues caused by varying blood pressure among patients. As used herein, the term “blood pressure” will mean either arterial pressure or pressure measured with a blood pressure cuff as is appropriate.

Another drawback of plunger-less syringes known in the art is that they employ materials that allow diffusion of the gases into or from the collected blood sample. That is, plunger-less syringes are generally made of a permeable polypropylene material that allows diffusion of gases. It has been conventional wisdom that for the short time periods in which a blood sample is retained in the receptacles of plunger-less syringes prior to testing that little or no gas would permeate within the body of the syringe. However, tests have shown that this assumption is not the case and that the porosity of polypropylene is such that in relatively short periods of time, i.e. 0-30 minutes, an appreciable amount of oxygen, for example, will diffuse between the interstitial boundaries that exist between the molecules of the material that make up the body of the plunger-less syringe. As stated above, maintaining the integrity of the blood sample is paramount. Any loss of gases from the blood sample into the plastic body of the plunger-less syringe or any gas introduced into the sample from an outside source would potentially adversely influence the accuracy of the blood gas analysis. Thus, there is a need for a plunger-less syringe that reduces the rate of diffusion of gases from or into a blood sample through the body of the syringe.

It is a further problem in collecting blood that the technician often collects a greater volume of blood than is needed for the test(s) to be conducted. Not only should the excess blood have remained in the patient for obvious reasons, but excess drawn blood creates an unnecessary disposal problem that is accentuated due to concerns for diseases, pathogens, etc. in the blood.

Thus it would be desirable to provide a plunger-less syringe wherein: (a) all or substantially all of the air in the blood collection receptacle is expelled as blood is being collected without altering the blood chemistry without adversely affecting the blood sample taken, (b) the material comprising the plunger-less syringe substantially prevents gases from diffusing into or from the blood sample; (c) the syringe has an exterior housing that is sized for ease of user handling while the blood collection receptacle therein is sized to accept only the volume of blood needed, and wherein the exterior housing and the collection receptacle are suitably secured together so that the entire syringe can be handled and/or stored with collected blood therein; (d) the components of the syringe are customizable to accommodate different patient characteristics, including different blood pressures; and (e) a legend or other indicia is associated with the syringe allowing the technician, nurse, doctor or healthcare provider to readily distinguish plunger-less syringes of the present invention configured for different situations, such as different blood pressures.

### SUMMARY OF THE INVENTION

The present invention includes a method and apparatus for obtaining blood from a patient, storing the blood obtained, and providing the obtained blood to a blood analysis instrument. In particular, the invention includes a plunger-less syringe (hereinafter “syringe”) particularly suited for collecting arterial blood, that may include an outer housing or tube with at least a portion of the outer dimensions of a conventional 3 cc syringe or otherwise having a common or uniform size easily handled and an interior receptacle or tube of a reduced size relative to the exterior for collecting blood. That is, an interior receptacle for receiving blood may be smaller and even substantially smaller than the outer housing. The receptacle or collecting volume may also be sized to correspond to the size of the sample needed. For example, a plurality of syringes may be produced corresponding to different collection volumes, and the exterior size may be uniform. In this manner the technician or nurse avoids removing more blood than necessary.

One embodiment of the present invention includes a barrel having a proximal end and a distal end and an internal space disposed between the two ends. A channel is positioned in the interior space and is in fluid communication with the distal end of the barrel. An insert is also provided that slidingly fits in the proximal end of the barrel. The insert includes a collection chamber or receptacle for receiving blood. An opening is integrated into to the proximal end of the insert that receives a filter that allows air to escape from the receptacle into the channel and out of an outlet positioned at the proximal end of the barrel. As blood is drawn into the receptacle air previously residing within the receptacle is forced through the filter and out through the channel. Preferably, and as described in greater detail below, a chemically treated filter may be employed such that once the receptacle is filled with blood and the filter becomes saturated with the blood, the filter seals to prevent air effectively exiting or entering the receptacle through the filter and to prevent blood from exiting the receptacle. In turn, the sealed filter prevents additional blood from entering the receptacle through the distal end of the insert or from exiting the receptacle. An effective seal is one that inhibits or prevents the flow of air under ambient conditions. If air pressure was increased sufficiently above ambient conditions, even though the filter is sealed, the increased pressure may cause air to flow through the filter. In this regard, some embodiments of the invention include an outlet at the distal end of the channel adapted to receive a plunger-type syringe, a bulb or similar pressure altering device to assist drawing blood from a patient or to push air through the channel and the filter (before it effectively seals) into the receptacle to expel the collected blood out of the distal end of the insert into a blood testing device. If the blood is being expelled from the receptacle for testing purposes, it may be desirable to displace the entire blood sample at once or to displace smaller volumes discretely, such as onto multiple slides.

It is another aspect of the present invention to provide a plurality of syringes that accommodate different patient physiologies or characteristics. More specifically, it is not uncommon for different patients to have different blood pressures. The patient's blood pressure is important and correlates to how easily blood enters the syringe and whether the blood flow rate will cause air residing within the receptacle to be trapped in the blood sample. More specifically, in individuals with high blood pressure, the blood is forced into the receptacle of the syringe at relatively high rates which can create localized low pressure areas that cause air to diffuse within the sample. Conversely, individuals with low blood pressure may have trouble filling the receptacle. Thus embodiments of the present invention are modifiable wherein barrels having air channels with smaller or larger volumes may be employed. A channel with a larger diameter may be used for individuals with low blood pressure. The larger diameter reduces impedance to the air entering receptacle that may be caused by the preexisting air in the channel. Conversely, for individuals with high blood pressure, the channel may be narrowed such that the air within a receptacle is impeded from transferring into the channel, which slows the flow of blood into the receptacle. In addition, one skilled in the art will appreciate that the receptacle diameter may be selectively increased or decreased to alter or throttle the flow of blood into the receptacle. Similarly, a throat or choke point may be positioned in the receptacle and/or channel that influences the air flow to achieve the same result. Further, the gauge size of the needle that may be interconnected to the inlet of the insert may be selectively altered to control the flow of blood into the syringe as is appropriate. Finally, the porosity of the filter may also be selectively altered, for example increased to account for individuals with low blood pressure or decreased to account for individuals with high blood pressure. All of these methods or combination thereof may be used to customize syringes for the physiology of different patients. A supply or inventory of syringes with different flow and/or volume characteristics may be stored and available for use by the technician or nurse. It is also contemplated that embodiments of the present invention employ color-coding or other indicators or indicia, such as words, symbols and colors or combinations thereof, to denote use of particular syringes with particular characteristics, such as different ranges of blood pressures and/or to denote the size of the blood collection receptacle. It is further contemplated that at least a portion of the exterior size and shape would remain consistent for ease of use by the technicians and nurses with the task of collecting and assessing the blood samples. Alternatively, the exterior of the syringe may be non-continuous and/or alterable, such as having a non-constant outer surface to accommodate blood receptacles of different sizes.

In some situations, bulbs or similar pressurizing devices may be utilized for generating a positive pressure within the receptacle, for example to assist in drawing blood from low blood pressure patients such as babies. This may be more advantageous than using the capillary action of syringe-less plastic or glass capillary tubes, particularly in the case of infants who do not typically lie still. The use of a bulb may quicken the blood collection time. For example, the devices described herein can easily fit in an individual's hand wherein the other hand may be used to hold a baby's heel. Positive pressure may be added via the bulb to thereby draw blood from the incision point. Thereafter, when the bulb is released, negative pressure is created that quickly draws additional blood into the collection receptacle. This method of fluid extraction also avoids air pockets being formed in the sample. In one embodiment, a 1,500 microliter volume bulb is added to the outlet of the barrel wherein the bulb is squeezed and then released to suction blood into the receptacle. It should be appreciated that a larger or smaller bulb may be used depending upon the context. For example, a larger bulb may be used that will allow for increased positive pressure generation that may be required to expel the collected blood, as described above, or if the volume of the blood receptacle is increased. One skilled in the art will appreciate that the bulb and stop cock or needle, if applicable, may be interconnected to the device by way of a luer slip or luer lock. Further, a bayonet interconnection scheme may be employed.

It is still yet another aspect of the present invention to help maintain the integrity of a blood sample. In addition to limiting the velocity of blood entering the receptacle it is also contemplated that the material surrounding the receptacle be constructed of a material that substantially prevents diffusion of the gases and/or to the blood from and/or to the blood sample therein. More specifically, addition of gas to, or removal of gas from the sample is detrimental and affects the result of the test. The molecular structure of the plastic material that comprises the insert is thus important. In the prior art, this material is generally formed of polypropylene, which is relatively permeable. As such, it has been found that existing blood collection inserts diffuse gas as a function of time, surface area of the receptacle, wall thickness of the insert, the blood volume, and the type of material employed. Regarding the material employed, it has been found that rigid polyvinylchloride (PVC) has a diffusion rate of one-tenth of that of polypropylene and that in addition, polyethylene terephthalate (PET) has a diffusion rate of one one-hundredth of that of polypropylene. PET is thus substantially non-permeable and non-diffusible relative to polypropylene wherein the gas dissolved in the blood has a diffusion rate less than 5% for a sample of 120 microliters.

It is another aspect of the present invention to provide a syringe that is compatible and is easily associated with commonly used blood analyzers. More specifically, it is desirous for obvious reasons, to quickly and easily transfer a blood sample from the syringe to the blood gas analyzer without spilling blood from the syringe. That is, it is desirable to ensure that the transfer of blood, which may contain various contaminants or pathogens, from the receptacle to the blood gas analyzer is seamless such that little or no blood is spilled or splattered on testing components, work surfaces or individuals. To prevent blood from escaping the syringe, often a seal or specialized filter is employed so that air cannot break the vacuum formed when the air was displaced from the receptacle during blood collection. Commonly, the specialized filter is comprised of a hydrophobic material impregnated with a chemical compound that forms a seal over time, which will be described below. Such filters may seal over a number of minutes depending upon the chemical used, the quantity of the chemical used and the filter material, such that a positive pressure greater than ambient is needed to force the blood out of the receptacle. Since the filter's sealing capacity increases over time, the positive pressure needed to extract the blood will necessarily increase with time. The pressure required to force the blood from the syringe is proportionate to increased occurrences of undesired splattering of the patient's blood.

Currently, there are two classes of blood testing machines 1) those that use positive pressure to force the blood into the blood gas analyzing machine, and 2) those that aspirate, i.e., siphon the blood from the syringe. For example, Abbott Laboratories produces the i-STAT 1™ handheld point of care analyzer. This analyzer requires a positive pressure wherein the blood is forced into the machine for analysis. Conversely, the ABL 80 Flex and ABL 77 of Radiometer automatically aspirate or suck the blood into the analyzer.

In a preferred embodiment, a filter positioned at the exit end of the receptacle is made of a hydrophobic material that contains a liquid reactive compound such as carboxyl methyl cellulose (CMC) that expands the filter material to close pores that otherwise would allow gas to pass. CMC is viscosity modifier or thickener commonly used in toothpaste, for example. When the blood contacts the CMC, it activates and either causes the filter material to expand or it fills the filter's pores to obstruct and/or prevent the transfer of gas through the filter. Obstruction of normal gas flow through the filter may take about 1 second or longer depending upon the quantity of chemical used and the pore size of the filter. However, the blocking of the filter pores occurs over time such that blood collected within the receptacle may be expelled from the syringe if the filter is exposed to positive pressure sufficient to force air through the restricted pores. That is, CMC or similar compounds are not immediately reactive, thereby enabling gas to continue to pass through the filter for a predetermined time until the pores are substantially blocked to prevent further gas flow. In this embodiment, the air within the receptacle would be displaced into the channel when the blood enters the receptacle. The blood would not be able to enter the channel due to the hydrophobic properties of the filter. However, it is also necessary to remove blood from the receptacle in order that it may be tested. Blood analyzing devices require that the blood either be aspirated or siphoned from the receptacle or forcibly expelled from and injected into the test device. Since some embodiments of the present invention utilize a filter that forms a seal after a predetermined time following exposure to blood, forced expulsion of the blood from the receptacle would be substantially impossible since the air that is required to displace the blood would be blocked by the sealed filter. As a result, some embodiments of the present invention may also employ a mechanism for disengaging the filter or otherwise breaking or overriding the seal it has created to allow air to enter the receptacle so that the blood may flow out of the receptacle. If the pores of the filter are not fully sealed, i.e., the blood sample has been delivered to the lab within an acceptable period of time, the channel and downstream filter may be exposed to positive pressure to force air through the filter in the opposite direction and as a result force blood from the receptacle. Alternatively, if the filter has become sealed, it is contemplated that the filter may be broken, punctured or otherwise circumvented in some way, for example, by a turn of the insert relative to the barrel, to effectively allow fluids and/or air to bypass the filter. In this situation, air would be forced or allowed to enter the receptacle so that blood could either be injected into a diagnostic machine or allowed to drip onto a test plate. Regardless, the mechanism permitting the expulsion of blood from the receptacle would also permit the exiting blood flow to be controlled.

In order to address the issue of having to provide increased positive pressure over time to transfer blood to the blood gas analyzer, one embodiment of the present invention employs a novel filtering/sealing scheme. More specifically, as described above, a hydrophobic filter that is impregnated with CMC will completely seal over time. Thus, in order to extract collected blood from the syringe, testing must be initiated relatively quickly after blood collection, i.e. no longer than a half of an hour. Further, in as little as five minutes increased positive pressure may be needed to force air through the pores of the filter. Thus, in order to allow the time period between blood collection and testing to be increased (but not necessarily increased to a point where detrimental gas diffusion or coagulation occurs), one embodiment of the present invention employs a dual filtering scheme, which will be described in further detail below. This filtering scheme is ideally suited for positive pressure machines. One of skill in the art will also appreciate that it may be employed with blood gas analyzers that use aspiration.

One embodiment of the present invention employs a dual filtering scheme involving a hydrophobic filter and a hydrophilic filter. The hydrophobic filter blocks the flow of liquid, but allows gas to pass. A hydrophilic filter allows liquid to pass but blocks the flow of gas after the filter is exposed to a liquid. When a hydrophilic material is exposed to liquid, the pores of the filter contract and the passage of gas is substantially restricted or blocked. However, by applying a sufficient pressure differential across the hydrophilic filter, gas can be forced through the filter. This pressure differential is often referred to as a “bubble point” of the filter, i.e., the pressure required to force air through the filter. One type of hydrophilic filters are made by Gore-tex®. It should be appreciated that hydrophilic filters are available with different characteristics. For example, the pore size may vary to block or allow passage of differently sized gas molecules. Similarly, hydrophilic filters are available with different bubble points. In addition, hydrophilic filters are available with pores that contract at different rates.

In comparison, hydrophobic filters employ appropriately sized pores to function as a barrier to the passage of liquids. The size of the pores may vary depending upon the filter material and the rate at which the pores restrict the flow of liquid may also vary depending upon the material. Systems containing dual filters are shown and described in U.S. Pat. No. 4,459,139 to von Reis et al., entitled “Disposable Filter Device and Liquid Aspirating System Incorporating Same” and U.S. Pat. No. 6,689,278 to Beplate, entitled “Combined Hydrophobic-Hydrophilic Filter for Fluids”, both of which are incorporated by reference herein.

In a dual filter arrangement, the filters may be adjacent one another, abutting or adhered to one another or spaced apart. Preferably, in one embodiment, the hydrophobic filter directly contacts the hydrophilic filter. Further, it is known that such filters may be joined in a single unit and obtained from a sheet of combined materials. In at least one embodiment of the invention a hydrophobic filter is placed on the exit end of the receptacle, between the barrel and the receptacle. A hydrophilic filter is placed adjacent to the hydrophobic filter, with the hydrophobic filter between the hydrophilic filter and the receptacle. The channel is on the other side of the hydrophobic filter. In operation, blood enters the receptacle and forces air from the receptacle through the hydrophobic filter. As the receptacle fills with blood, the blood contacts the hydrophobic filter. The air displaced from the receptacle will also pass through the hydrophilic filter, enter the channel, and then exit the syringe. One skilled in the art will appreciate that the flow of blood can thus be controlled somewhat by altering the pore size of the hydrophilic filter. The larger the pore size of the hydrophilic filter, the greater the mass flow rate possible of the gas/air traveling from the receptacle into the channel. Conversely, if the pore size of the hydrophilic filter is restricted, air will not easily be transferred from the receptacle to the channel, thus slowing the flow of blood into the receptacle. Thus, the combination of filters may be used to selectively alter the intake of a blood sample.

When the blood reaches the hydrophobic filter, most if not all of the air has been transferred out of the receptacle. It is important to note, that in this embodiment of the present invention, preferably, the hydrophobic filter is not impregnated with the CMC or similar material that causes the hydrophobic filter to block both liquids and gases over time. The hydrophobic filter of one embodiment possesses a pore size that allows a sufficient amount of fluid flow therethrough so that the hydrophilic filter can be saturated. That is, hydrophobic filters have a water-breakthrough point, i.e., the amount of pressure differential across the filter required to drive fluid therethrough. Blood will saturate the hydrophobic filter and, thus, will necessarily contact the adjoining or abutting hydrophilic filter. In combination, both filters will effectively block the transmission of blood and gas therethrough. Blood is prevented from exiting the receptacle by the hydrophilic filter as it prevents air from transitioning from the channel back into the receptacle. In this respect, the hydrophilic filter operates similar to the CMC impregnated hydrophobic filter of the prior art. This configuration has the advantage of allowing blood to be forced from the receptacle for periods longer than would be possible when a CMC impregnated hydrophobic filter is used. In addition, the saturated hydrophobic filter prevents a great amount of additional blood fluid from entering into the receptacle, stopping the blood withdrawal process because the blood cannot freely pass through the hydrophobic filter. Importantly, blood will not flow out of the receptacle due to gravity because the hydrophilic filter is blocking the flow of air into the receptacle, effectively placing a cap on the receptacle, provided the ambient air pressure is less than the bubble pressure of the hydrophilic filter.

To extract the fluid from the receptacle, sufficient positive pressure may be added into the channel to force air through the hydrophilic filter and through the hydrophobic filter to force the blood through the inlet of the syringe. More specifically, if the pressure differential across the hydrophilic filter exceeds the bubble point of the filter (i.e. the pressure required to force air through the filter), fluid can be forced out of the receptacle. Caution should be exercised because the introduction of air at the required pressures may cause diffusion of excess air into the blood. The pressure may also cause the blood to exit the receptacle at a rate faster than desired, causing a loss of control of the blood flow. Alternatively, the hydrophilic filter may be pierced, broken, or the seal it has created otherwise circumvented thereby allowing air to travel from the channel through the hydrophobic filter and into the receptacle which allows the blood to be extracted from the syringe. Further still, an aspirating instrument may be used to suck blood from the receptacle without circumventing the seal created by the hydrophilic filter.

One skilled in the art will appreciate that the order or position of the hydrophobic and the hydrophilic filters may be switched. For machines that use positive pressure exclusively to obtain a blood sample, the hydrophilic filter may be placed between the receptacle and the hydrophobic filter. Thus, in operation, as the blood is drawn within the receptacle it would displace the air in the receptacle through the hydrophilic filter and the hydrophobic filter. The air would then move into the channel. When the receptacle is filled with blood, the hydrophilic filter would become saturated and thus allow blood to contact the hydrophobic filter. At that point, all of the air that was originally within the receptacle will have been transferred through the channel and out of the syringe. Air is prevented from moving from the channel into the receptacle by the hydrophilic filter, where air restricting characteristics have been activated by contact with the patient's blood. To remove the blood, the hydrophilic filter, which is contact with the blood, may be circumvented, but this may be a difficult task since the hydrophilic filter will be in contact with the blood. Methods and structures are nonetheless disclosed herein for accomplishing this. Alternatively, the channel may be exposed to increased positive pressure, i.e. higher than the bubble point of the hydrophilic filter. Circumventing the air seal created by the filters is not an issue for aspiration-type blood analyzers.

A related embodiment of the present invention employs a hydrophobic filter (no CMC) and a cap positioned on the outlet or proximal end of the syringe. More specifically, as blood is collected, air from the receptacle will pass through the hydrophobic filter. Once the receptacle is filled, a cap is placed on the outlet of the syringe, thereby preventing the additional transfer of air between the receptacle and the channel. As long as the cap remains in place, the collected blood is maintained within the receptacle. This mechanism is akin to holding one's finger over the open end of a straw and pulling it from a cup of water. The water from the straw is prevented from exiting due to the difference of the pressure between the fluid within the straw and the gas within the straw. The inlet of the syringe may also be selectively blocked. Further still, a selectively openable aperture in the body of the syringe may be used to facilitate air movement allowing for the collection and evacuation of blood. The aperture would be open during the blood collection process and would be closed to prevent blood from escaping once collection was complete. To remove the blood, one would simply open the aperture or remove the cap to allow air to travel through the hydrophobic filter. Further, if a cap is used, the cap may employ a luer lock, valve or other type of device that selectively allows movement of air within the channel.

It is another aspect of the present invention to provide a valve that is selectively interconnected or integrated into the syringe. Preferably, this valve would be associated with the outlet of the syringe and operate as the cap described above. The valve may be a flapper valve or a valve that is selectively opened by pinching, such as found on air mattresses, for example. Further still, an adhesive seal or tape may be positioned across the proximal end of the syringe to temporarily block air flow and thereby prevent the blood from exiting the receptacle. At the appropriate time the seal or tape may be physically removed. Generally, any mechanical valve is also contemplated that would selectively allow air to enter and exit the channel to control the flow of blood.

It is yet another aspect of the present invention to employ an anticoagulant coating such as heparin (highly-sulfated glycosaminoglycan) is used in conjunction with embodiments of the present invention. Often, collected blood will coagulate within the collection device between the time of collection and the time it is tested. Thus embodiments of the present invention include an anticoagulant coating applied to at least a portion of the inner surface of the receptacle. Preferably, the coating is sputtered or blown into the walls of the receptacle or other known application methods are used to place the anticoagulant in the receptacle. The coating may be a powder or a liquid.

Alternatively to coating the internal portions of the insert, embodiments of the present invention employ a mixing ball positioned within the insert to help prevent coagulation. As briefly described above, the internal geometry of the insert may be altered to accommodate varying sizes of blood samples to be taken. Additionally, the internal geometry may include a relatively consistent internal cross-section such as the cylinder or be conical in nature, for example. With respect to the use of a mixing ball, this conical configuration is preferred since the location of the mixing ball can be controlled such that is does not block the distal end of the insert and it prevents the mixing ball from falling out of the syringe. The mixing ball is used to excite the stored fluid sample prior to entering the testing device. The ball need not be spherical, but any of a variety of shapes would work equally well. The use of the term “ball” is not limited to a spherical shape.

It is yet another aspect of the present invention to employ a mixing ball that has been coated with an anticoagulant material. More specifically, embodiments of the present invention employ a mixing ball that has been treated with a fixed amount of anticoagulant material, such as heparin. The amount of anticoagulant used may be based upon a ratio of the surface area of the mixing ball, the solution concentration of the anticoagulant and the volume of blood to be withdrawn. Thus each mixing ball would have a consistent quantity of anticoagulant. It is contemplated that the mixing ball may have a generally smooth surface or be porous to help absorb and maintain the anticoagulant coating.

In one embodiment, the anticoagulant may be added to the mixing ball by placing the balls of known weight and surface area into a bath of anticoagulant solution wherein the ball sinks to a predetermined depth. The solution will be lyophilized (freeze-dried) about the ball and thus bonded thereto. Preferably, the ball will be covered over more than one-half of the surface area of the ball such that the heparin will be securely interconnected thereto and less susceptible to being dislodged. Using this method, a portion of the surface of the ball would not be exposed to the bath leaving a surface area available to manipulate the ball. Such manipulation could be accomplished by vacuum or other known means to situate and remove the balls to and from the bath. One skilled in the art will appreciate a porous ball would also have the ability to be placed in a bath of anticoagulant such that the solution would travel via capillary action within its interior. Other methods are contemplated such that a ball could be placed in a container such that a lower surface thereof is sealed. A fixed amount of anticoagulant would then be added to the container such that the upper surface of the ball is exposed. The result would be a ball with a stripe of bonded anticoagulant.

Other features and benefits of the present invention will become evident from the description herein below together with the accompanying drawings.

The Summary of the Invention is neither intended nor should it be construed as being representative of the full extent and scope of the present invention. The present invention is set forth in various levels of detail in the Summary of the Invention as well as in the attached drawings and the Detailed Description of the Invention and no limitation as to the scope of the present invention is intended by either the inclusion or non-inclusion of elements, components, etc. in this Summary of the Invention. Additional aspects of the present invention will become more readily apparent from the Detail Description, particularly when taken together with the drawings.

### BRIEF DESCRIPTION OF THE DRAWINGS

The accompanying drawings, which are incorporated in and constitute a part of the specification, illustrate embodiments of the invention and together with the general description of the invention given above and the detailed description of the drawings given below, serve to explain the principles of these inventions.

FIG. 1 is an exploded perspective view of a syringe of one, embodiment of the present invention;

FIG. 2 is a cross sectional view of FIG. 1;

FIG. 3 is a sectional view of a barrel shown in FIG. 1;

FIG. 4 is a front elevation view of an insert of one embodiment of the present invention;

FIG. 5 is a cross-sectional view of FIG. 4;

FIG. 6 is a sectional view of an alternate embodiment of the insert;

FIG. 7 is a sectional view of an alternate embodiment of the insert;

FIG. 8 is a right cross-sectional view along line 8-8 of the insert shown in FIG. 7;

FIG. 9 is a perspective view of an alternate embodiment of the syringe;

FIG. 10 is an exploded cross-sectional view of FIG. 9;

FIG. 11 is a cross-sectional view of FIG. 9;

FIG. 12 is a cross-sectional view of an alternative embodiment of a collection receptacle employed by the embodiment shown in FIG. 9;

FIG. 13 is a cross sectional view of a mixing ball positioned in a bath of anticoagulant material;

FIG. 14 is a mixing ball showing a coating of anticoagulant material applied thereto;

FIG. 15 is a cross-sectional view of an alternative embodiment of the present invention wherein a dual filtering system is employed on the syringe shown in FIG. 1;

FIG. 16 is a cross-sectional view of an alternative embodiment of the present invention wherein a dual filtering system is employed on the syringe shown in FIG. 9;

FIG. 17 is a detail view of FIG. 16;

FIG. 18 is a detail view of FIG. 16 wherein a third portion of the syringe is shown in a second position of use;

FIG. 19 is a detail view of FIG. 16, showing an alternative method of bypassing a filter;

FIG. 20 is a detail view of FIG. 16, showing an alternative method of bypassing a filter wherein a the third portion of the syringe is shown in a second position of use;

FIG. 21 is a view showing the extraction of blood using the syringe of one embodiment in conjunction with a needle;

FIG. 22 is a view showing the extraction of blood using the syringe of one embodiment in conjunction with an intravenous catheter;

FIG. 23 is a front elevation view showing a plungered syringe interconnected to the syringe of one embodiment of the present invention;

FIG. 24 is a view showing a syringe associated with a blood gas analyzing machine;

FIG. 25 is a cross-sectional view of an insert of another embodiment of the present invention; and

FIG. 26 is a cross-sectional view of the insert shown in FIG. 25, wherein a spacer is shown located within the receptacle.

It should be understood that the drawings are not necessarily to scale. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

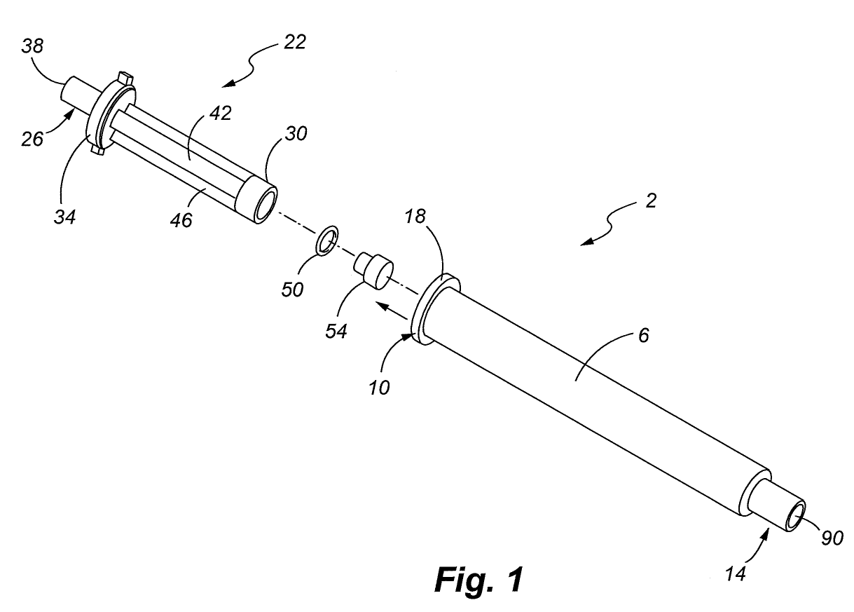

Referring to FIG. 1, a syringe 2 is provided that includes the barrel 6 with a distal end 10 and a proximal end 14 with a rim 18 extending outwardly from the distal end 10. An insert 22 is also shown that includes a distal end 26 and an outlet 30. A rim or energy rib 34 extends outwardly from the distal end 26 of the insert 22. The distal end 26 of the insert 22 includes and inlet 38 adapted to interconnect with a needle or other known device for purposes of collecting blood. A receiving chamber or receptacle 42 is positioned between the inlet 38 and the proximal end 30 of the insert 22 and is adapted to receive and hold fluids such as blood. A flange or rib 46 may be provided that extends the length of the receptacle 46 to provide some rigidity to the insert 22. The proximal end 30 of the insert 22 is adapted to receive a seal 50 and a filter 54. In one embodiment, the filter 54 is designed to allow gas to exit the receptacle 42 through the filter 54 but to prevent liquids from exiting from the receptacle 42 through the filter 54. In one embodiment, the filter is impregnated with carboxyl methyl cellulose (CMC), a welling agent that closes the pores so that the blood collected in the receptacle 42 sample remains anaerobic and cannot pass through the filter. Preferably, if only a portion of the filter includes CMC material, that portion of the filter is oriented away from the blood collecting receptacle if possible. Optionally, a cap or other type of closing device may be fitted on the distal end 26 of the insert to prevent collected blood from exiting the inlet 38.

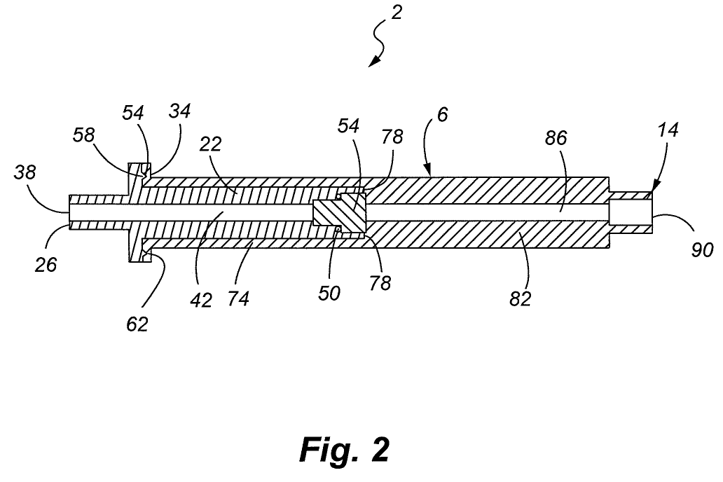

Referring now to FIG. 2, an assembled syringe 2 shown in FIG. 1 is provided. Here, the syringe 2 includes an outer configuration similar to that of a 3 cc syringe commonly used, which provides familiarity to the user and aids in syringe handling. It should be appreciated that different sizes and shapes may be used. The rim 34 of the insert 22 and the rim 18 of the barrel 6 are adapted to be sealed together by known methods such as ultrasonic welding, adhesives and/or an interference fit, etc. The interfacing surfaces of the two rims may also be provided with alignment or mating features such as a protrusion 58 extending from one surface and a corresponding aperture or groove 62 in the other surface. Ultrasonic welding is a process that employs an acoustic tool to transfer vibration energy through to the weld area. The friction of the vibrating molecules of the protrusion on the rim of the barrel generates heat that melts the plastic to weld the barrel and insert together. One skilled in the art will appreciate that the syringe may also be of one-piece construction.

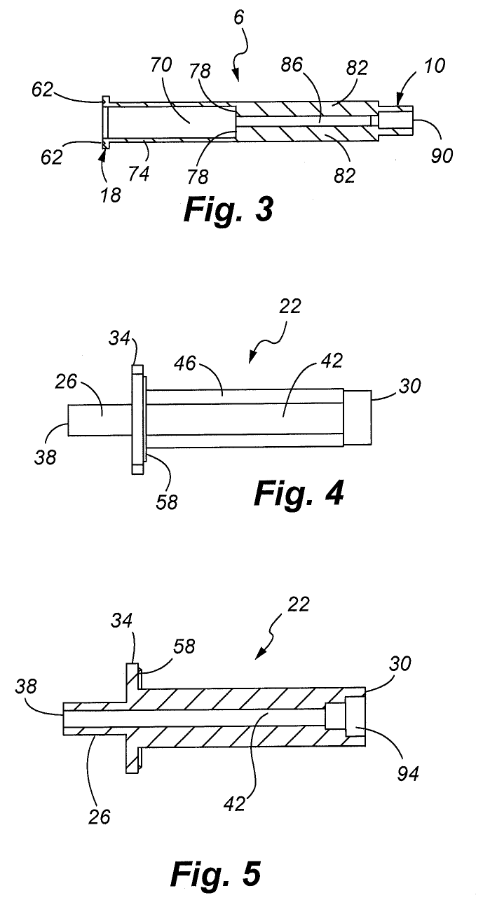

Referring now to FIG. 3, the barrel 6 of one embodiment of the present invention is provided. More specifically, the barrel 6 has a distal end 10 which defines an internal volume 70 in which the insert 22 is adapted to fit. The volume of the internal volume 70 is defined by the thickness and axial length of wall 74. Thus, the internal volume 70 may be adjusted to accommodate inserts 22 of varying dimensions. With the insert 22 positioned in the internal volume 70 as shown in FIG. 2, the proximal end 30 of the insert abuts a shoulder 78 formed by wall 82 of the barrel 6. As shown in FIG. 3, the wall 82 is thicker than the wall 74. The wall 82 further defines a channel 86 extending from the internal volume 70 to the outlet 90 formed in the proximal end 14 of the barrel 6, Accordingly, the inlet 38 formed a the distal end 26 of the insert 22 is in fluid communication with the outlet 90 formed at the proximal end 14 of the barrel 6. The outlet 90 is adapted to interconnect with a number of devices which will be described in further detail below, including a syringe with a plunger, a bulb, a valve, a seal or caps of various configurations.

The dimensions of channel 86 may be selectively altered to control the rate at which the blood enters the receptacle 42. Controlling the blood flow rate can substantially reduce, assist or encourage blood flow in patients with low blood pressure and slow blood flow in patients with high blood pressure. In the latter situation, substantially reducing the amount of turbulent flow present in the blood flow as it enters the receptacle 42 advantageously maintains the integrity of the gas content within the blood sample. The outer diameter of the barrel 6 may be any diameter that is ergonomically comfortable to technicians and nurses, etc., but is preferably that of 3 cc syringe known in the art.

Another advantage provided by making receptacles 42 of a known volume is that the volume can be set to match the size of the blood sample utilized in testing equipment, thereby limiting the volume of blood removed from a patient to no more than utilized in the prescribed test. In this manner, excess blood does not need to be disposed of, eliminating a biological hazard issue, and more importantly in the mind of the patient, more blood remains in the patient's body. Thus a variety of receptacle sizes may be available to technicians and/or nurses when drawing blood to match the receptacle size to the prescribed test or tests to be performed. Alternatively, in some embodiments the position of the insert 22 relative to the barrel 6 may be adjusted relative to each other to enlarge or reduce the volume of the receptacle 42 dynamically, rather than utilizing a syringe with a fixed volume receptacle 42 as shown in other embodiments. This may be accomplished, for example, via a threaded or sliding interconnection. It is envisioned that sealing means known by those skilled in the art would also be used to ensure that no blood would escape from the receptacle in these embodiments.

FIGS. 4 and 5 show the insert 22 provided by one embodiment of the present invention. As discussed above, the insert 22 includes the distal end 26 and a proximal end 30 wherein the syringe inlet 38 is located adjacent to the distal end 26. Situated adjacent to the proximal end 30 of the insert 22 is a cavity 94 for receipt of the seal 50 and filter 54. The filter may be any shape and, if desired, is adapted to create a barrier between the receptacle 42 and the channel 86. The cavity 94 and filter 54 define an opening into the receptacle 42 on the opposite end of the receptacle 42 from the inlet 38. As blood is collected in the receptacle 42 air previously in the receptacle is forced out of the receptacle 42 through the filter 54 and into the channel 86. With reference to FIGS. 1-5, the channel 86 as shown is of constant diameter but one skilled in the art will appreciate that the diameter may be selectively altered to dictate the flow characteristics of the blood being received within the receptacle 42. More specifically, one skilled in the art will appreciate that the diameter of the channel 42 may be increased or decreased relative to the receptacle exit defined by the cavity 94 and the filter 54 such that a throat is created by shoulders 78 to decrease the pressure of the air exiting the receptacle 42 through the filter 54. By increasing the diameter of the channel 86, a pressure drop will occur at the entrance to the channel 86 and air will exit the receptacle more quickly thereby allowing for blood to enter the receptacle more quickly. Conversely, this configuration may be changed such that it is more difficult for air to exit from the receptacle 42 into the channel 86 thereby slowing the flow of blood into the receptacle 42 to accommodate individuals with high blood pressure.

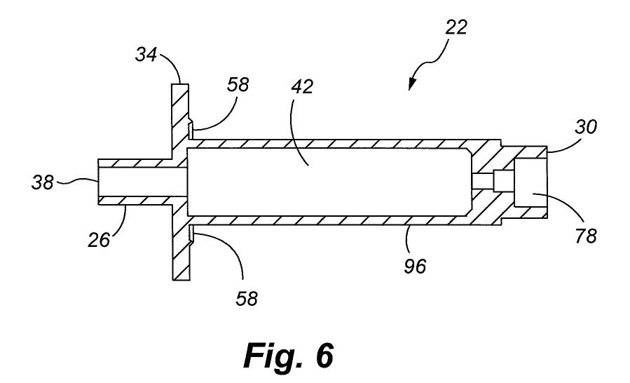

Referring now to FIG. 6, an alternate embodiment of the insert 22 is provided. More specifically, in order to alter the volume of collected blood the channel 86 volume may be expanded or contracted. Here, the channel 86 is expanded substantially such that the thickness of its wall 96 is relatively thin. This configuration is somewhat easier to manufacture than the embodiment with ribs 46 described above and designed so that the outer diameter of the insert 22 substantially coincides with the inner diameter of the internal volume 70 of the barrel 6.

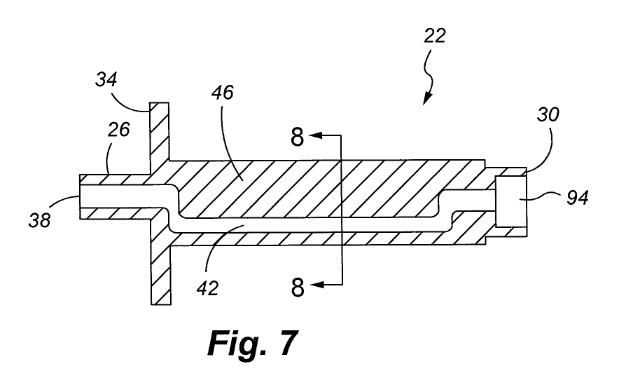

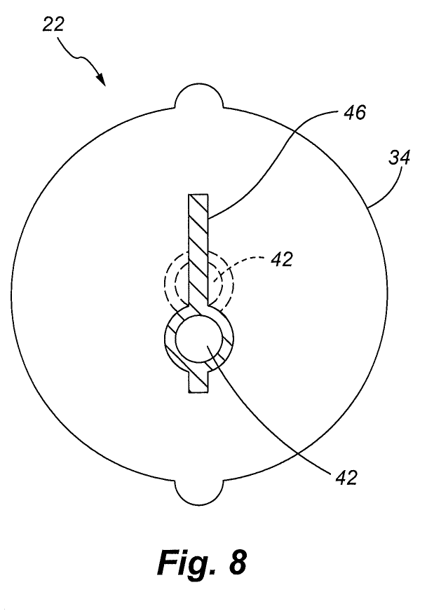

Referring now to FIGS. 7 and 8, yet another configuration of the insert 22 is provided wherein the receptacle 42 is not situated within the center of the insert 22, but is positioned offset from the longitudinal center axis thereof. Here, the flange 46 is enlarged to accommodate the offset location of the receptacle 42. This configuration also increases the manufacturability of the product. One skilled in the art will appreciate that various other alterations may be employed to enhance manufacturability and/or vary the fluid flow characteristics of within the insert 22. The embodiment shown employs a non-linear receptacle 42 that prevents the incoming blood from prematurely contacting the filter. That is, a non-direct path from the inlet 38 to the filter cavity 94 is contemplated so that the blood received from a patient with high blood pressure, for example, will not immediately contact the filter, which would prematurely activate the CMC and affect the filter's ability to transfer air therethrough.

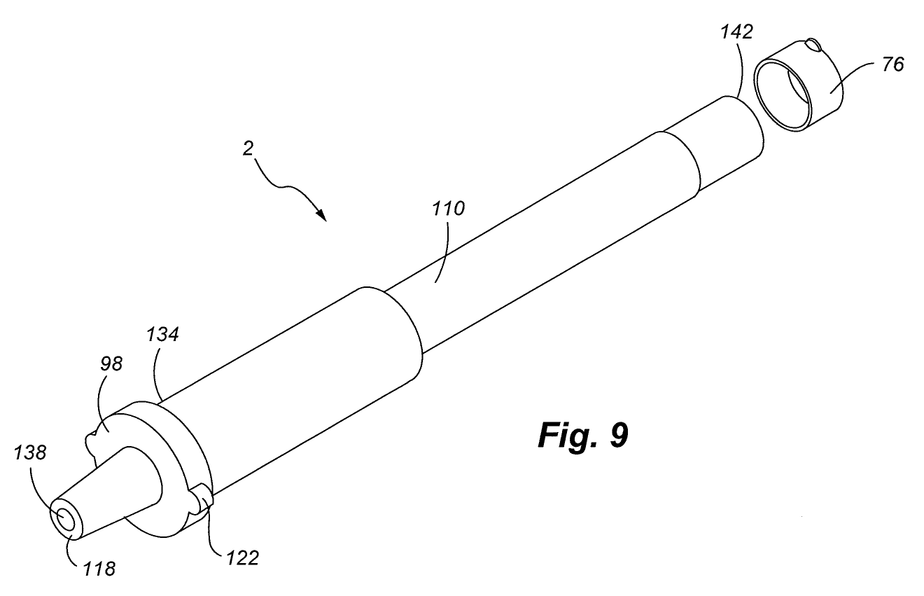

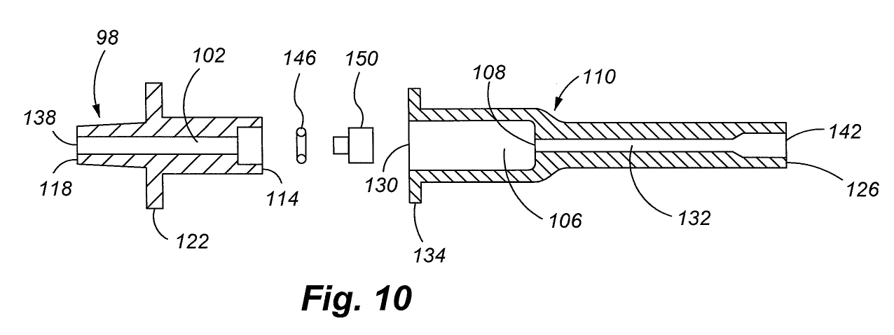

Referring now to FIGS. 9-12, another embodiment of the present invention is shown. Here, a first member or insert 98, which includes a receptacle 102, is adapted to be received by an internal volume 106 of a barrel or second member 110. The first member further includes a proximal end 114 and a distal end 118. A rim 122 extends outwardly from the first member 98. The second member includes a proximal end 126 and a distal end 130. The second member includes a channel 132 that is in fluid communication with an internal volume 106 formed at the distal end 130 of the second member. A rim 134 is also included that extends from the second member 110 that is adapted to engage the rim 122 of the first member 98.

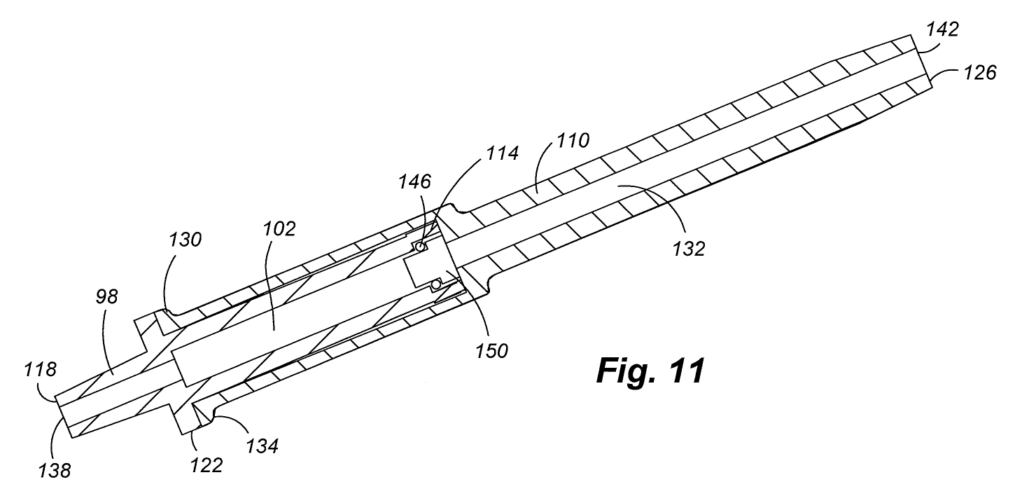

Referring specifically to FIG. 11, when the first member 98 and the second member 110 are interconnected, a syringe 2 is formed having an inlet 138 and an outlet 142. In addition, an o-ring 146 or other sealing means is associated with the first member 98 and a filter 150, which functions similar to what has been described above.

As described above, this embodiment of the present invention also allows the size of the receptacle 102 and/or channel 132 to be selectively altered in order to compensate for a patient's blood flow or to capture a precise or limited volume of blood. More specifically, the size of the receptacle 102 and/or the size of the channel 132 may be selectively altered to vary the rate of blood flow in the receptacle or to limit the volume of blood withdrawn from a patient, for example, to match the volume needed for test purposes, without taking more. Further, the filter 150 may also be altered in composition to control the flow of blood within the receptacle 102. For example, the size of the pores may be increased or decreased and the material from which the filter is made may be changed to create different flow rates. In addition, a second filter may also be associated with the filter 150 as described above. One skilled in the art will appreciate that although a slidable engagement between the first member 98 and the second member 110 is alluded to, other interconnection schemes, such as a threaded interconnection may be used. Preferably, the first member 98 and the second member 110 are interconnected via a ultrasonic weld associated with rim 122 and rim 134. However, other interconnection schemes such as bayonet fittings, luer locks, etc., as known to those of skill in the art, may be employed without departing from the scope of the invention.

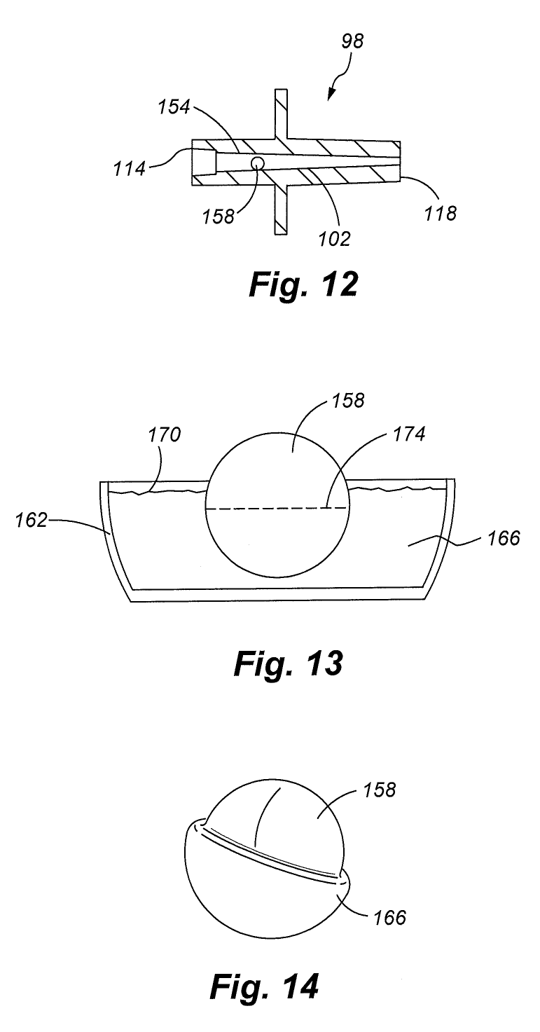

Referring now to FIG. 12, an alternative embodiment of a cross-section of the first member 98 is shown. Here, the receptacle 102 includes a sidewall 154 wherein a portion thereof is conical or shaped in various other ways to control the location of a mixing ball 158 positioned therein. This shape prevents the mixing ball from exiting the distal end 118 of the first member 98.

Referring now to FIGS. 13 and 14, a mixing ball 158 of one embodiment of the present invention is shown. Here, the mixing ball 158 is placed in a liquid bath 92 containing an anticoagulant material 166. In one embodiment, the mixing ball 158 is generally spherical and is placed in the anticoagulant bath 162 such that the mid-line 174 of the ball is below the level 170 of the bath 162. The ball 158 is then removed and the coating is subsequently freeze-dried to harden the anticoagulant material onto the ball 158. Preferably, the anticoagulant material will reside above the mid-line 174 of the ball 158 to provide a mechanical bond between the ball 158 and the anticoagulant material 166 after it has been dried thereto. It is also envisioned that the ball 158 may be added to and removed from the bath 162 via a suctioning or vacuum means that engages the uncoated or upper portion of the ball 158. One skilled in the art will appreciate that many ways may be used to apply the anticoagulant material to the mixing ball 158. One skilled in the art will also appreciate that the quantity of anticoagulant attached to the ball may vary depending upon the methodology used. It may be desirable to have more or less anticoagulant in order to match the volume of the withdrawn blood or to vary the effects based upon the time the sample will be held prior to testing. It should further be appreciated that the ball need not be spherical but could be a variety of other shapes, provided that it adequately carries the anticoagulant. The anticoagulant may also be applied as a coating on the surface of the inner walls of the receptacle, with or without a mixing ball or carrier.

Embodiments of the present invention may employ a system wherein the filter is made of a hydrophobic material allowing air but not liquid to pass. As the blood enters the receptacle it will come in contact with the filter and will be prevented from moving thereby. The air originally stored within the receptacle will be allowed to pass through the filter and into the channel. Because an air seal is not created, the blood sample may be expelled from the receptacle without breaking the seal. However, this is also somewhat problematic because air in the channel can also re-enter the receptacle and disrupt the flow of blood or permit the blood to prematurely exit the receptacle, such as following extraction and prior to testing.

Syringes of the prior art address this problem by impregnating the hydrophobic filter with CMC, thereby creating a static seal after a given time after the hydrophobic material is exposed to blood. To obtain access to the collected blood using an injection-type blood analyzer, a positive pressure must be applied to the hydrophobic material to force air from the channel into the receptacle. However, this is not an issue when using an aspiration technique to remove blood for testing wherein a needle is placed within the inlet of the receptacle and is adapted to pull blood therefrom.

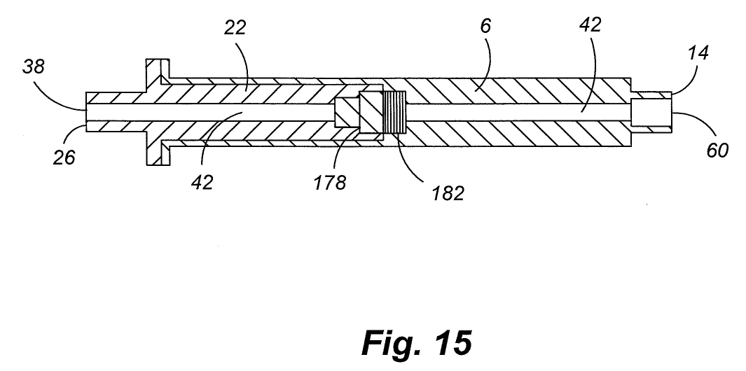

Referring now to FIGS. 15-20, to address this issue, embodiments of the present invention may employ a hydrophobic filter 178 positioned adjacent to or in an abutting relationship with a hydrophilic filter 182. The hydrophilic filter 182 allows liquids to pass but inhibits the flow of air once the filter is wetted. In operation, as blood enters the receptacle 42 it pushes air through the hydrophobic filter 178 and then through the hydrophilic filter 182. As described above, saturation of the hydrophobic filter 178 substantially prevents the syringe from receiving further blood into the receptacle 42 because blood is prevented or limited from passing through the hydrophobic filter 178. In addition, due to the proximity of the hydrophilic filter 182 to the hydrophobic filter 178, the hydrophilic filter is exposed to blood, thereby activating the hydrophilic filter 182 to restrict the flow of air. As a result, the hydrophilic filter 182 prevents gas from re-entering from the receptacle 42, thereby preventing the blood captured within the receptacle 42 from escaping under ambient or normal gravitational circumstances. To remove blood from the receptacle 42, the user would break, or otherwise circumvent the hydrophilic filter 182 opening an air passageway through the hydrophobic filter 178. One skilled in the art will appreciate that a pressurization instrument, such as a plungered syringe or bulb, may also be interconnected to the outlet 60 and used to pull a negative pressure to suction the blood into the receptacle 42. The pressurization instrument may also be maintained on the device after blood collection to maintain blood within the receptacle and to provide a positive pressure to force the fluid from the receptacle 42, for example, by overcoming the bubble pressure of the hydrophilic filter.

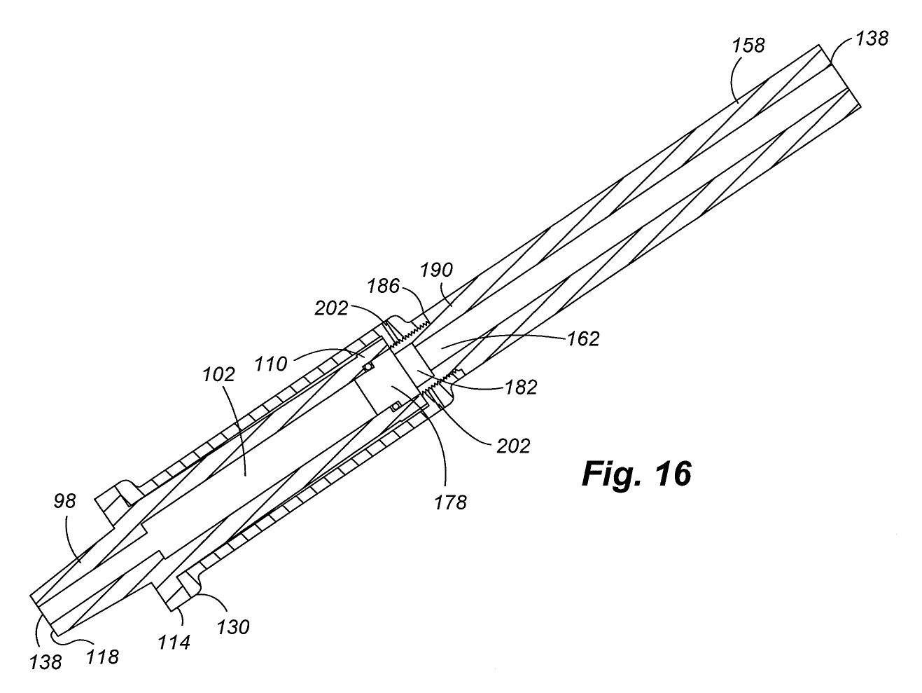

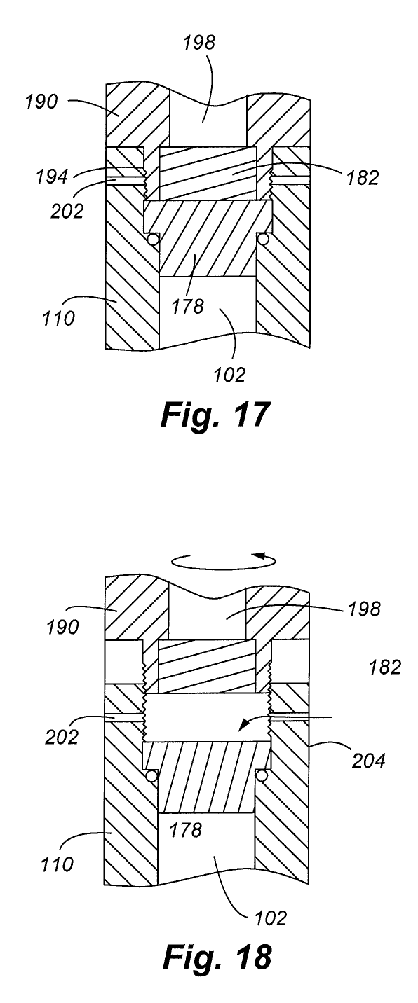

Referring now specifically to FIGS. 16-20, a method of selectively circumventing the hydrophilic filter 182 is provided. The embodiment of FIG. 16 generally shows the configuration of the embodiment of FIG. 11 wherein a first member 98 is interconnected to a second member 110. The first member includes a rim 122 that is interconnected, preferably by an ultrasonic weld, to a rim 134 of the second member 110. Alternatively, the first and second members may be a single molded piece, such as is shown in FIGS. 17-20. The assembled syringe 2 thus includes an inlet 138 and an outlet 142. The first member 98 also includes a receptacle 102 for receiving blood. A sealing mechanism, such as an o-ring 146 is placed between the hydrophobic filter 178 and the receptacle 102 to ensure that blood does not escape from the receptacle 102. It should be appreciated that the o-ring or seal 146 may be positioned at other locations as known by those of skill in the art, for example at any location in the fluid pathway where compression holds the filters in place.

Referring specifically to FIG. 16, in addition to a first member 98 and a second member 110, a third member 190 is included that is interconnected to the second member 110 via a joint or connection point 186. The third member 190 and the second member 110 may be ultrasonically welded together to form the joint 186, or preferably may utilize threads 166 to interconnect the second member 110 to the third member 190. A hydrophilic filter 182 is positioned within the third member 190 and adjacent to the hydrophobic filter 178. The two filters may be in physical contact with each other or have a small air gap or space between them. The third member 190 also includes an internal volume or air passageway 198. Further, an air channel 202 is integrated into the end of the second member 110. The air channel 202 allows air to circumvent the hydrophilic filter 182 through the wall 204 of the second member 110.

In operation, blood is prevented from exiting the receptacle and into the channel 198 by the hydrophobic filter 178. Air originally residing within the receptacle 102 is transferred into the channel 198. The hydrophilic filter 182 is designed to inhibit air flow once exposed to a liquid, such as blood, and thereby prevent air from re-entering the receptacle 102. Even though the hydrophobic filter 178 would ideally halt all blood flow, in some instances a quantity of blood will seep through the hydrophobic filter 178 in small but sufficient amounts to adequately contact and activate the air flow restriction characteristics of the hydrophilic filter 182. In addition, the relative positioning of the second member 110 and third member 190 block the air channels 202. In order to allow air to re-enter the receptacle 102, thereby allowing blood to be removed from the receptacle 102 by gravity, the third member 190 is moved relative to the second member 102 to open the air channels 202, for example by unscrewing the third member a sufficient amount such that the third member no longer blocks the air channels 202. Providing a relatively small space between the two filters may assist in moving the third member 190 relative to the second member 110. It will be understood by one skilled in the art that although two air channels 202 are provided, a single air channel or a plurality thereof may be employed without departing from the scope of the invention. In FIGS. 17 and 18 the same relative movement opens the air channels 202, although in these embodiments, the first and second members are formed as a single component piece 110 with a wall 204.

In addition, one skilled in the art will appreciate that the air channels 202 may be omitted and instead, the threads 194 allow for the transmission of air therethrough from the outside environment when loosened. Further, a slot or groove may be incorporated into the threads 194, wherein non-continuous threads are provided such that in one position no continuous groove or slot is formed and air cannot pass and upon a relative repositioning of the second members 110 and third member 190, the slots in the threads are aligned to allow air from the outside environment to circumvent the hydrophilic filter 182. Further, the third member 190 may be designed to be completely removed from the second member 110 and reused on another syringe. Interconnection between the third member 190 and the second member 110 may be made a way of a luer lock as described by U.S. Pat. No. 4,369,781 to Gilsen et al., entitled “Luer Connector,” which is incorporated by reference in its entirety herein. Luer connectors are well known in the art and any type of such may be used without departing from the scope of the invention. It is also contemplated that the third member 190 may be separated from the second member 110 by cracking an ultrasonic weld of the joint 186, thereby creating an air channel circumventing hydrophilic filter 182.

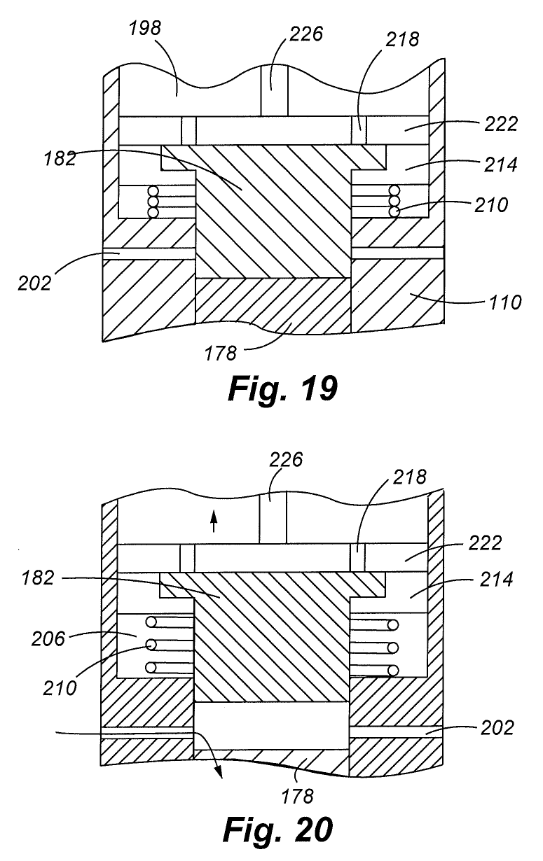

Referring additionally to FIGS. 19 and 20, yet another method of circumventing the hydrophilic filter 182 is provided. To illustrate the many ways the hydrophilic filter 182 may be circumvented, a mechanism analogous to those found in retractable pens is shown. Here, the second member 110 includes an increased bore 206 positioned adjacent to the outlet 142. This bore 206 receives a spring 210 with a filter seat 214 positioned thereon. The filter seat 214 provides a location for seating the hydrophilic filter 182. At least one air channel 202 is integrated into the second member and a plunger 222 is utilized that rests on the filter seat 214 and the filter 182. The plunger has passages 218 to let air transition through the hydrophilic filter 182 into the channel 198 that is positioned aft of the plunger 222 toward the proximal end of the syringe when blood is being drawn into the receptacle 102. The position of the plunger is controlled by a post 226. During blood collection, air channels 202 are blocked.

In operation, the post 226 transitions the plunger 222 as commonly found in a pen, wherein in a first position, the spring 210 is compressed and the hydrophilic filter 182 is seated in the filter seat 214 blocks the air channel. In the first position the hydrophilic filter 182 is placed generally adjacent to or in contact with the hydrophobic filter 178 or with a relatively small space between the two filters. In this first position of use, air can transition through the hydrophilic filter 182 and through either the air channel 202 or the passage 218. In a second position of use, shown in FIG. 20, the post 226 transitions the plunger 222 upwardly, thereby displacing the filter seat and the hydrophilic filter 182. When the hydrophilic filter 182 is transitioned away from the hydrophobic filter 178 and towards the outlet 142 of the syringe 2, the air channel 202 is exposed, thereby allowing air to enter therethrough and into the receptacle 94. When air enters the receptacle 94, the collected blood is allowed to exit the receptacle 102. To stop the flow of blood from the receptacle 102, the post 226 is pushed downwardly to cause the hydrophilic filter 182 to block openings 202. As a further alternative, it is also possible to move both filters simultaneously to open air flow channels 202 rather than moving only the hydrophilic filter.

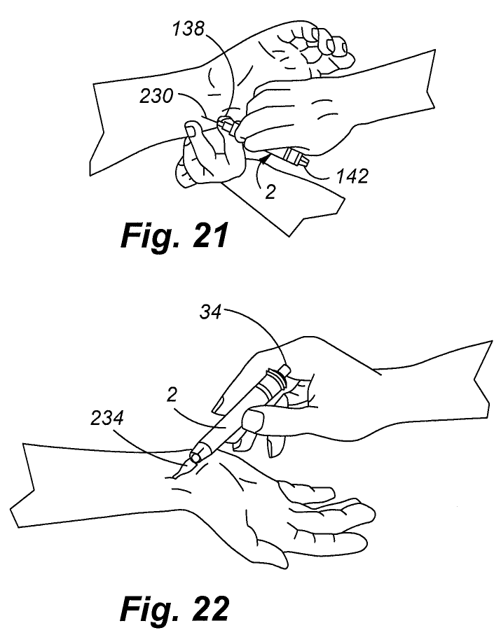

Referring now to FIGS. 21-24, a method of extracting blood from a patient for analyzing the same is shown. Here, the syringe 2 is interconnected to a needle 230. This interconnection may be made of any commonly known method, such as luer locks which utilize threads, for example. The needle 230 is then placed into an artery of a patient and the blood pressure of the patient allows the blood to enter into the syringe 2. In the case of a child, the syringe 2 may be placed adjacent to a small incision.

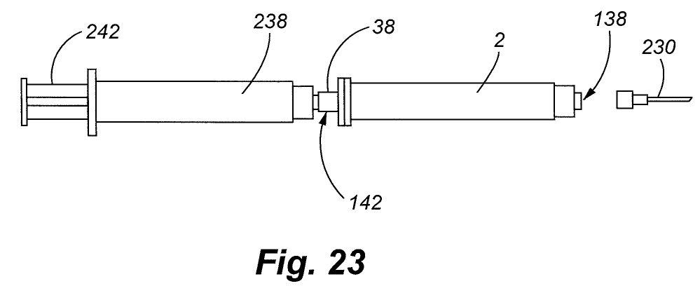

Referring now to FIG. 23, a plungered syringe 238 is shown interconnected to the syringe 2 of embodiments of the present invention. More specifically, the plungered syringe is inserted into the outlet 142 of the syringe 2. In addition, a needle 230 may be interconnected to the inlet 138 of the syringe 2. In order to collect blood from the patient, a healthcare provider would pull on a plunger 242 to create a negative pressure within the plungered syringe 238 to draw blood in the syringe 2. Blood may be extracted from the syringe 2 by removing the needle 230 and associating the inlet 138 with a blood analyzing device, which will be described in detail below. If the blood analyzing device is a positive pressure machine, the plunger 242 may be transitioned back to a starting location to create positive pressure in the syringe 2 to force the blood therefrom.

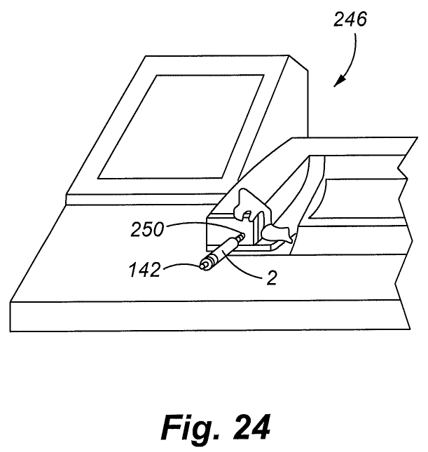

Referring now to FIG. 24, a blood analyzing device 246 is shown that includes an orifice 250 for the receipt of the syringe 2. In the event positive pressure is required, a syringe 2 or other means may be interconnected to the outlet 142 of the syringe 2.

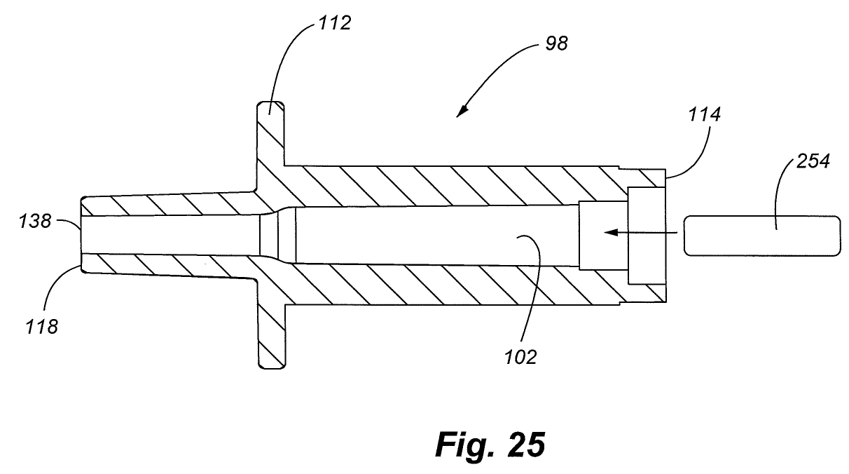

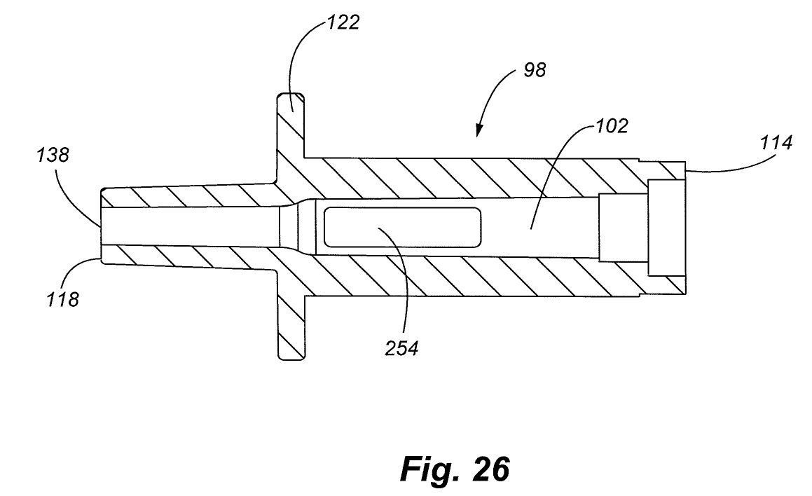

Referring now to FIGS. 25 and 26, another embodiment of the insert 98 is shown that possesses a receptacle 102 that is adapted to receive a spacer 254 that selectively alters the volume of the receptacle 102. FIG. 25 shows the insert 98 with a spacer 254 positioned adjacent thereto, and FIG. 26 shows the spacer 254 positioned in the receptacle 102 which reduces the volume thereof. Thus, this embodiment employs a receptacle 102 of a single volume that is selectively reduced by the addition of at least one spacer 254, which omits the need to manufacture and supply syringes or inserts of varying volumes. The spacers 254 may be any shape and may be marked with indicia that instructs the technician the relative size of the spacer and/or the resulting volume of the receptacle 102 that will be provided if the spacer 254 is used. The indicia may be words, a numeric coding, a color coding or a combination of these three. Thus, for example, for an insert 98 with a 500 milliliter receptacle 102, multiple spacers may be provided in varying sizes, such as 100, 200 and 300 milliliters. Of course, other sizes could be provided. The spacers 254 may be made of any plastic or metal that would not alter the integrity of the blood sample, and may also be provided with an anti-coagulant coating and function as a mixing ball described above. The spacers are shaped, for example as a rectangle or other appropriate shape, or otherwise provided with passageways extending through the entire spacer to prevent the spacer from blocking the flow of fluids, including air and blood. Alternatively, physical barriers may be designed into the body of the collection receptacle to prevent the spacer from blocking fluid flow.

While various embodiments of the present invention have been described in detail, it is apparent that modifications and alterations of those embodiments will occur to those skilled in the art. However, it is to be expressly understood that such modifications and alterations are within the scope and spirit of the present invention, as set forth in the following claims. Further, one skilled in the art after review of the foregoing will appreciate that the function and the arrangement of the barrel 6 and insert 22 and first member 98 and second member 100 are interchangeable. For example, the barrel/second member may be used to collect blood and the insert/first member may be used to receive air displaced by the collected blood.

The foregoing discussion of the invention has been presented for purposes of illustration and description. Further, the description is not intended to limit the invention to the form disclosed herein. Consequently, variation and modification commensurate with the above teachings, within the skill and knowledge of the relevant art, are within the scope of the present invention. The embodiment described hereinabove is further intended to explain the best mode presently known of practicing the invention and to enable others skilled in the art to utilize the invention as such, or in other embodiments, and with the various modifications required by their particular application or uses of the invention.

What is claimed is:

1. A device for collecting blood, comprising:
a first member having a receptacle adapted for collecting and holding blood, said receptacle including means for preventing coagulation of the collected blood;
a second member having a fluid inlet, a fluid outlet and a channel therebetween, said channel in fluid communication with said receptacle, said channel adapted to receive air displaced from said receptacle when blood is introduced thereto; and
a means for restricting fluid flow positioned between said first member and said second member that allows for the passage of air from said receptacle to said chamber and prevents blood from passing from said receptacle to said chamber.

2. The device of claim 1, wherein said fluid outlet of said second member is adapted to receive a means for changing the pressure of the air in said second member.

3. The device of claim 2, wherein said means for changing the pressure is at least one of a plunger, a syringe with a plunger, and a bulb.

4. The device of claim 3, wherein said bulb has a volume greater than or equal to the volume of said receptacle.

5. The device of claim 2, wherein said means for changing pressure is adapted to create a negative pressure to draw fluid into said receptacle and is adapted to provide positive pressure to force collected fluid from said receptacle.

6. The device of claim 1, wherein said first member is made of at least one of a rigid polyvinylchloride and a polyethylene terephthalate.

7. The device of claim 1, wherein said means for preventing coagulation of blood is highly-sulfated glycosaminoglycan.

8. The device of claim 1, wherein said first member is ultrasonically welded to said second member.

9. The device of claim 1, wherein said first member includes an opening with a first rim protruding therefrom and said second member includes a second rim spaced distally from said fluid outlet, the first rim and the second rim being adapted for interconnection.

10. The device of claim 9, wherein said first rim includes a protrusion that engages said second rim and is ultrasonically welded thereto.

11. The device of claim 1, wherein said means for preventing coagulation of blood is a mixing ball positioned within said receptacle.

12. The device of claim 11, wherein the walls of said receptacle comprise a conical portion.

13. The device of claim 12, wherein said mixing ball includes a coating of anticoagulant material.

14. The device of claim 13, wherein said mixing ball is not spherical.

15. The device of claim 1, wherein said means for preventing coagulation of blood is an anticoagulant material positioned within said receptacle.

16. The device of claim 1, further comprising indicia associated with said device, said indicia corresponding with a blood pressure.

17. The device of claim 16, wherein the blood pressure is arterial pressure.

18. The device of claim 1, further comprising indicia associated with said device, said indicia corresponding with the volume of blood that may be stored in said receptacle.

19. The device of claim 16, wherein said indicia is at least one of a symbol and a color.

20. The device of claim 16, wherein said indicia appears on at least one of said first member, said second member and said means for restricting flow.

21. The device of claim 1, wherein at least one of said first member, said second member and said means for restricting flow are of a color that corresponds with the blood pressure of a patient.

22. The device of claim 1, wherein said means for restricting fluid flow is a filter made of a hydrophobic material.

23. The device of claim 22, further comprising:
a third member having an air channel therein, said third member selectively interconnected to said fluid outlet of said second member, wherein in a first position said air passageway is blocked such that air cannot pass through said air passageway and in a second position said air passageway is opened to allow air to enter said channel of said second member.

24. The device of claim 23, wherein said second member is interconnected to said third member by way of a threaded interconnection.

25. The device of claim 23, wherein said second member is interconnected to said third member by way of a luer interconnection.

26. The device of claim 1, further comprising a second means for restricting fluid flow positioned between said first means for restricting flow and an open end of said second member.

27. The device of claim 26, wherein said second means for restricting flow is a filter made of a hydrophilic material.

28. The device of claim 1, further comprising a cap that is adapted for interconnection to said second member and is at least one of selectively adjustable and removable to allow extraction of blood.

29. The device of claim 1, wherein said first member comprises a fluid inlet through which blood travels when entering said receptacle, and further comprising a cap adapted for interconnection to said fluid inlet to prevent the blood from exiting said receptacle through said fluid inlet.

30. The device of claim 1, wherein said first member is a barrel having a proximal end and a distal end with an outer wall therebetween that defines an internal volume, the proximal end having an opening that provides access into the internal volume, the distal end having a fluid inlet, the barrel further including said receptacle adapted to receive and store blood and disposed between the inlet and the internal volume, and an insert adapted to be positioned within the internal volume of the barrel, the insert having a proximal end with a fluid outlet and a distal end having an opening with a channel positioned therebetween, said channel in fluid communication with said receptacle.

31. The device of claim 1, wherein said first member includes a proximal end and a distal end with an outer wall therebetween that defines said receptacle for the receipt of blood, and wherein said second member includes a proximal end and a distal end with an outer wall therebetween that defines an internal volume for receiving said first member and a fluid channel disposed between said receptacle and said proximal end of said second member, said channel in fluid communication with said receptacle.

32. The device of claim 31, further comprising a hydrophilic filter positioned in said channel of said third member.

33. The device of claim 1, wherein said means for preventing coagulation of collected blood is a spacer positioned in said receptacle.

34. The device of claim 1, wherein said means for preventing coagulation of collected blood is a plurality of differently sized spacers individually positionable in said receptacle.

35. The device of claim 34, wherein said plurality of spacers comprise an anticoagulant material.

36. The device of claim 34, further comprising indicia associated with each of said plurality of spacers that provide volumetric information.

37. A method of limiting coagulation of a blood sample, comprising:
placing a mixing ball in a bath of an anticoagulant;
covering a portion of the surface area of the mixing ball with anticoagulant;
solidifying the anticoagulant on the mixing ball;
placing the mixing ball in the blood receiving portion of a plunger-less syringe;
using the syringe to draw blood from a person;
contacting the drawn blood with the mixing ball containing anticoagulant.

38. The method of claim 37, wherein removing said mixing ball from said bath of anticoagulant comprises moving the mixing ball with a vacuum-assisted device.

39. The method of claim 37, wherein solidifying the anticoagulant comprises freeze-drying.

40. A system for drawing blood from patients for testing purposes, comprising:
providing a first plurality of syringes having a receptacle for receiving and holding blood, and having a first throat to regulate the flow of blood into the receptacle, said throat corresponding to a first blood pressure;
providing a second plurality of syringes having a receptacle for receiving and holding blood, and having a second throat to regulate the flow of blood into the receptacle, said second throat corresponding to a second blood pressure; and
indicia associated with said first and second plurality of syringes for indicating the blood pressure each corresponds to and for distinguishing said first plurality of syringes from said second plurality of syringes.

41. The system of claim 40, wherein said first and second plurality of syringes were plunger-less syringes.

42. The system of claim 40, wherein said first and second blood pressures comprise a range of blood pressures.

43. The system of claim 40, wherein said indicia comprises at least one of a color and a symbol.

44. The system of claim 40, further comprising providing a third plurality of syringes having a receptacle for receiving and holding blood, and having a third throat to regulate the flow of blood into said receptacle, said throat corresponding to a third blood pressure, and indicia associated with said third plurality of syringes for indicating the blood pressure corresponding to the third plurality of syringes and for distinguishing said third plurality of syringes from said first and second plurality of syringes.

45. The system of claim 40, wherein said first and second throats comprise at least one of a passageway and the pores of a filter.

46. The system for drawing blood of claim 40, wherein said first and second blood pressures are arterial pressures.

47. The method of manufacturing a plunger less syringe, comprising:
providing a first member having a receptacle for receiving blood;
positioning a mixing ball within said receptacle;
providing a second member having a channel integrated therein that cooperates with said receptacle;
positioning first filter between said receptacle and said channel; and
interconnecting said first member to said second member.

48. The method of claim 47, wherein said second member is inserted into said first member.

49. The method of claim 47, wherein said first member is inserted into said second member.

50. The method of claim 47, further comprising adding an indicia to at least one of said first member, second member, and said filter, said indicia indicative of a characteristic of the plunger-less syringe.

51. The method of claim 50, wherein the characteristic of said plunger-less syringe is the flow characteristics of the blood as it is drawn from a patient.

52. The method of claim 47, wherein said first member is interconnected to said second member with an ultrasonic weld.

53. The method of claim 47, wherein said receptacle comprises a conical shape.

54. The method of claim 47, further comprising adding an anticoagulant material to at least one of said mixing ball and said receptacle.

55. The method of claim 54, wherein said anticoagulant material is added to said mixing ball by placing said mixing ball into a bath of said anticoagulant material such that a portion of said mixing ball is coated and a portion of said mixing ball remains uncoated.

56. The method of claim 55, wherein said mixing ball is removed from said bath by a vacuum process.

57. The method of claim 47, further comprising positioning a second filter in said second member on the side of the first filter opposite said receptacle.

58. The method of claim 57, wherein said filter is hydrophobic and said second filter is hydrophilic.

59. The method of claim 47, wherein said mixing ball is provided in a plurality of sizes to alter the volume of said receptacle.

60. A system for drawing blood from patients for testing purposes, comprising:
providing a first plurality of syringes having a receptacle for receiving and holding blood, said receptacles being of substantially the same size in each syringe; and
providing a second plurality of spacers of different physical sizes, said spacers adapted to be inserted into the receptacle of a syringe to alter the volume of blood said receptacle may hold.

61. The system of claim 60, further comprising indicia associated with said spacers for differentiating spacers of different sizes.

62. The system of claim 60, further comprising indicia associated with said spacers for indicating at least one of the volume of said spacer and the resulting volume of said receptacle when said spacer is positioned in said receptacle.

63. The system of claim 60, wherein said plurality of syringes are plunger-less syringes.

64. The system of claim 60, wherein said indicia comprises at least one of a color and a symbol.

65. The system of claim 60, where in said spacers further comprise an anti-coagulating material.

66. The system of claim 60, wherein said spacers further comprise a fluid passageway through the body of said spacers.

67. The system of claim 60, further comprising means disposed in said syringe for preventing said spacer from blocking the flow of fluids in said syringe.
